(19) United States
(12) Patent Application Publication
(10) Pub. No.: US 2007/0293773 A1
(43) Pub. Date: Dec. 20, 2007

(54) Arm Support Device Particularly For Use In An Apparatus For Measuring Arterial Pressure

(76) Inventor: Francesca Ghigini, Milano (IT)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 11/791,245

(22) PCT Filed: Nov. 15, 2005
(86) PCT No.: PCT/EP05/55997

§ 371 Date: May 21, 2007

(30) Foreign Application Priority Data
Nov. 25, 2004 (IT) MI2004A002283

(51) Int. Cl. A61B 5/02

(52) U.S. Cl. 600/485

(57) ABSTRACT

“A device (1) for supporting the arm of a patient in order to measure blood pressure, which includes a pedestal (2) provided with a support (4) for resting the arm and a mechanism (7) adapted to detect the height of the patient and a mechanism (9, 10, 11) adapted to move the support and arrange it vertically as a function of the detected height.”

### TECHNICAL FIELD

The present invention relates to an arm support device particularly for use in an apparatus for measuring arterial pressure.

### BACKGROUND ART

As it is known, arterial pressure is normally measured by using a sphygmomanometer generally constituted by a pressure gauge connected to an air chamber which can be inflated by means of a bulb. The instrument is designed to oppose a known pressure to the arterial pressure and to allow the reading of the pressure values when the flow of blood is detected by stethoscopic auscultation during the decompression of the cuff provided with the air chamber.

The air chamber, integrated in the cuff applied to the arm of the patient, produces on the arm a pressure which, at a certain point of compression, exceeds the arterial pressure, interrupting the flow of blood downstream of the cuff.

Once the arterial pressure has been exceeded by 20-30 mm/Hg, the cuff is decompressed by means of a pneumatic valve incorporated within the bulb.

During decompression, the operator listens to the sounds generated by the artery by using a stethoscope which is appropriately rested against the arm.

In this manner, he detects a series of sounds of different intensity, duration and tone, which are generated by the arterial pulses, which in turn are produced by cardiac activity and by the resistance of the arterial vessel. The operator must establish which of these pulses represents the systolic value and which one represents the diastolic value of the pressure.

An essential condition for the reliability of the measurement is that the pulse detection point must be at the same height as the heart of the patient.

It is in fact known that in any hydraulic circuit which operates in a dynamic condition with a pressure source, the value of the pressure is influenced by the difference in level between the pump and the point being tested.

In the case of arterial pressure measurement, considering the specific gravity of blood (1055 g/l), the error caused by the difference in level is ±760 torr (mm/Hg abs)/cm²×1055/1000=0.8018 mm/Hg per centimeter of difference in level. Therefore, a difference in level of 10 cm produces an error of approximately 8 mm/Hg.

In medical practice, the difference in level is normally not checked or is checked roughly, by resting the arm of the patient, for practical reasons, always on the same surface, regardless of the height of the patient.

On the other hand, checking the position is not easy to do, since the difference in level can be determined by several variables or points:

height and posture of the subject;

level of the seat;

level of the point where the forearm of the patient rests.

An error due to difference in level can compromise correct diagnosis and the consequent therapeutic decision if one considers that, according to the indications of the World Health Organization, the difference between the upper level of the normal value and the value of first-degree hypertension is 5 mm/Hg.

Moreover, apart from the damage which can be caused to a patient by giving him a treatment for hypertension when he is not at all affected by hypertension, one must also consider the economic damage to the subject and/or to the community caused by the prescription of unnecessary treatments.

### DISCLOSURE OF THE INVENTION

The aim of the present invention is to provide an arm support device for use in an apparatus for measuring arterial pressure, in which the position of the arm of the patient can be adjusted according to the height of the patient.

Within this aim, an object of the present invention is to provide a device for supporting the arm of the patient for an apparatus for measuring arterial pressure, in which the resting point of the arm of the patient can be adjusted easily and automatically.

Another object of the present invention is to provide a device for supporting the arm of the patient which is highly reliable, relatively simple to manufacture and at competitive costs.

This aim and these and other objects which will become better apparent hereinafter are achieved by a device for supporting the arm of a patient in order to measure blood pressure, which comprises a pedestal provided with a support for resting the arm, characterized in that it comprises means adapted to detect the height of the patient and means adapted to move said support and arrange it vertically as a function of said detected height.

### BRIEF DESCRIPTION OF THE DRAWINGS

Further characteristics and advantages of the present invention will become better apparent from the following detailed description of a preferred but not exclusive embodiment of the device according to the present invention, illustrated by way of non-limiting example in the accompanying drawings, wherein:

FIG. 1 is a side elevation view of the device according to the invention;

FIG. 2 is a sectional view of a detail of the device according to the invention.

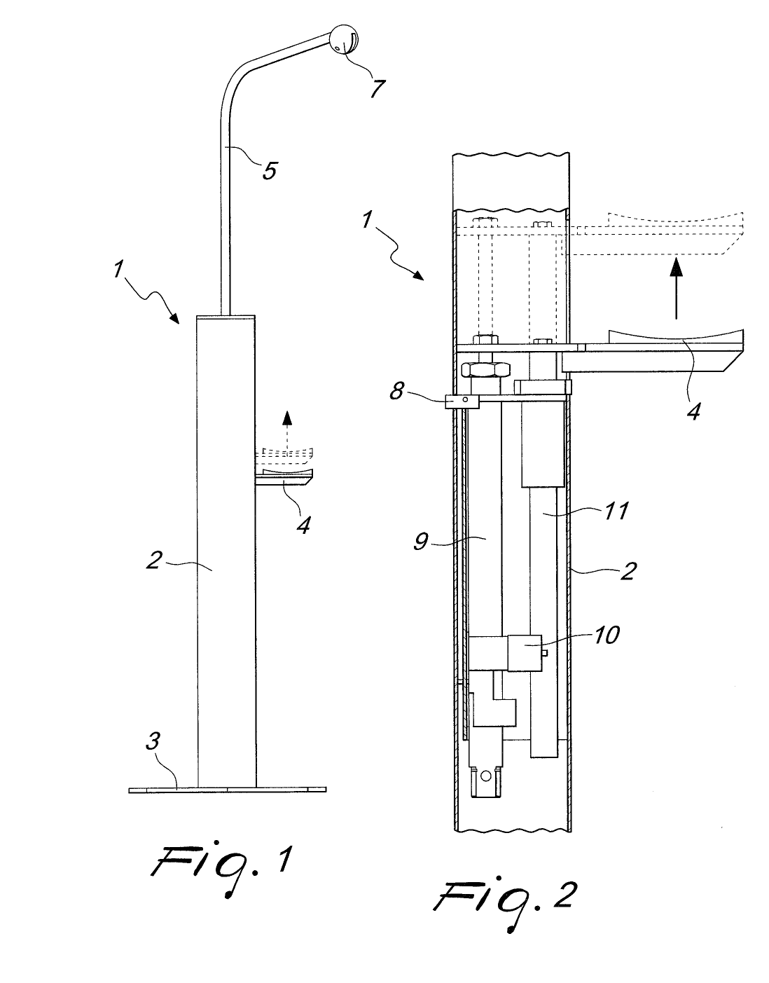

### WAYS OF CARRYING OUT THE INVENTION

With reference to the figures, a device according to the present invention, generally designated by the reference numeral 1, comprises a pedestal, which is constituted by a tubular element 2 provided with a base 3 and with a support 4 which allows to rest the arm of the patient thereon. Conveniently, the support 4 can be adjusted in its height with respect to the ground according to the height of the patient who rests his arm on the support 4.

Moreover, the device has a rod-like element 5, at the end of which a height detection device 7 is provided, which is constituted by a ultrasound source and an ultrasound receiver. When speaking of height, what is meant here is the distance between the top of the head of the patient and the sitting surface on which the patient is sitting.

The height detector is adapted to command the movement of means for lifting/lowering the support 4 for the arm of the patient.

Conveniently, the movement means for lifting/lowering the support 4 comprise at least one compressor 10, which is adapted to actuate a pneumatic cylinder 9 which slides along a cylinder movement guide 11. The movement of the support 4 is measured by means of a photocell 8 and the graduated rod.

The supporting element 4 is rigidly coupled to the pneumatic cylinder 9 and therefore the movement of such cylinder allows to lift/lower the support 4 with respect to the plane on which the base element 3 of the pedestal rests.

Substantially, when the patient rests his arm on the support 4, the height of the patient is detected, automatically or on command of the operator, by means of the ultrasound source and the corresponding receiver, and the support 4 is positioned automatically at the correct height, i.e., at the heart of the patient, so that the measurement of the pressure is free from the error caused by any difference in level between the height of the heart and the height of the arm.

Therefore, the device according to the invention allows to adapt substantially instantaneously the height of the support 4 for the arm of the patient depending on the detected height of the patient, so as to align the support 4 at the height of the heart in order to be able to obtain a blood pressure measurement which is error-free at least as regards the difference in level between the height of the heart and the height of the arm.

Substantially, the “height” information acquired from the patient is processed by means of an algorithm which determines from it the height that the armrest must assume. It is possible to construct experimentally or by means of anthropomorphic data (which can be variable among different populations) a correlation curve between height and heart position and therefore the chosen position of the armrest.

In practice it has been found that the device according to the invention fully achieves the intended aim and objects, since it allows to eliminate one of the causes of error in pressure measurement which can lead to the diagnosis of hypertension or to underestimate the pressure values.

The device thus conceived is susceptible of numerous modifications and variations, all of which are within the scope of the appended claims; all the details may further be replaced with other technically equivalent elements.

In practice, the materials used, as well as the contingent shapes and dimensions, may be any according to requirements and the state of the art, without thereby abandoning the scope of the protection of the appended claims.

The disclosures in Italian Patent Application No. MI2004A002283 from which this application claims priority are incorporated herein by reference.

1. A device for supporting an arm of a patient in order to measure blood pressure, the device comprising a pedestal provided with a support for resting the arm, characterized in that the device comprises means adapted to detect a height of the patient and means adapted to move said support and arrange the support vertically as a function of said detected height.

2. The device according to claim 1, characterized in that said means adapted to detect the height of the patient comprise an ultrasound source and an ultrasound receiver.

3. The device according to claim 2, characterized in that said ultrasound source and said ultrasound receiver are arranged at an end of a rod-like element which protrudes from said pedestal.

4. The device according to claim 1, characterized in that said means adapted to move said support comprise a compressor which is suitable to actuate a pneumatic cylinder which is rigidly connected to said support, said pneumatic cylinder being guided along a movement guide.

5. The device according to claim 4, characterized in that the device comprises a graduated rod which is arranged along said pedestal, said pneumatic cylinder being adapted to slide vertically along said graduated rod.

6. A method for determining a correct height of an armrest for supporting an arm of a patient in a blood pressure measurement apparatus, characterized in that the method comprises the steps of:
sitting the patient on a seat of said apparatus;
detecting the distance between a top of a head of the patient and a sitting surface of said seat; and
on the basis of the detected distance, positioning the armrest of said apparatus so that the arm of the patient is at the same height as a heart of the patient.

7. The method according to claim 6, characterized in that the positioning of said armrest is performed by analyzing a correlation curve which links the measured height information to the height of the heart of the patient and therefore to the position of the armrest with respect to a seat on which the patient is sitting.
